(19) United States
(12) Patent Application Publication
(10) Pub. No.: US 2026/0263286 A1
(43) Pub. Date: Sep. 10, 2026

(54) DEVICE FOR DRESSING WOUNDS AND METHODS OF USE THEREOF

(71) Applicant: Tiger Wound Care Medical, LLC, Conshohocken, PA (US)

(72) Inventors: Lucas FORNACE, Plano, TX (US); Jeff GREINER, Plano, TX (US)

(21) Appl. No.: 19/532,914

(22) Filed: Feb. 6, 2026

Related U.S. Application Data
(60) Provisional application No. 63/929,563, filed on Dec. 2, 2025.
(60) Provisional application No. 63/768,644, filed on Mar. 7, 2025.

(51) Int. Cl. A61F 13/05; A61F 13/00; A61F 13/02; A61L 15/44

(52) U.S. Cl. CPC A61F 13/05; A61F 13/00063; A61F 13/0243; A61L 15/44; A61F 2013/0017; A61F 2013/00285; A61F 2013/00536; A61F 2013/00859; A61F 2013/00863; A61M 2202/0415; A61M 2202/0427; A61M 2202/045

(57) ABSTRACT

The present disclosure features devices, kits, and methods for providing dressings and uses thereof for localized and contained application of a biomaterial to a subject (e.g., the skin and/or a wound) for medical and/or cosmetic purposes. The devices disclosed herein can be used to promote drying and coagulation of the biomaterial, thereby forming a biomaterial dressing that remains applied upon removal of the wound dressing.

### PRIORITY

The present application claims priority to U.S. Provisional Patent Application Nos. 63/768,644, filed Mar. 7, 2025, and 63/929,563, filed Dec. 2, 2025, the entire disclosures of which are incorporated herein by reference.

### BACKGROUND

Wound healing is a significant focus of the medical field, as treatments are being developed to address various types of wounds, such as surgical wounds, lacerations, abrasions, burns, and traumatic injuries. Various materials, chemical and biological, are used for this purpose and several systems have been developed to ensure effective delivery of wound healing agents to the different layers of the skin and tissue.

Thus, there exists a need for devices and methods for producing an effective biomaterial dressing which improve the distribution and conformity of biomaterial on the wound.

### SUMMARY OF THE DISCLOSURE

The present disclosure features devices, kits, and methods for applying dressings and uses thereof for localized and contained application of a biomaterial to a subject (e.g., the skin and/or a wound) for medical and/or cosmetic purposes. The wound dressing disclosed herein can be used to promote drying and/or coagulation of the biomaterial, thereby forming a biomaterial dressing. The wound dressing disclosed herein may be applied to the wound before applying the biomaterial to facilitate placement of the biomaterial. The application of the biomaterial may be performed with an attached injection port to allow attachment of a container, such as a syringe. The wound dressing may also allow for ventilation of the wound during and following an application of the biomaterial. The wound dressing may further be separable to allow removal of components, such as the injection port to provide comfort to the subject as the biomaterial solidifies and/or the wound heals. The biomaterial may be capable of remaining applied to skin even upon removal of the wound dressing.

A first aspect of the present disclosure is directed to a wound dressing for applying a biomaterial to a subject, the wound dressing comprising: a first layer comprising a first opening and a plurality of first pores having a first width; and a second layer comprising a second opening and one or more second pores having a second width, wherein the first layer is configured to be between the second layer and the subject when the wound dressing is applied to the subject, the first opening is at least partially aligned with the second opening to allow passage of the biomaterial through the first layer and the second layer, and the second width is larger than the first width.

In some embodiments, the wound dressing of the first aspect may include one or more of the following features. At least a subset of the first pores may be at least partially aligned with the one or more second pores to allow for ventilation through the first layer and the second layer. A distal surface of the second layer may be at least partially uncovered to allow for ventilation through the second layer directly to the atmosphere. The one or more second pores may be uncovered distally and directly open to the atmosphere. The first width may be from about 0.2 microns to about 500 microns, inclusive. The second width may be from about 1 mm to about 10 mm, inclusive. The first layer may be a mesh. The mesh may be non-absorbent. The wound dressing may include a valve configured for one-way passage of the biomaterial through the first opening and/or the second opening. The valve may be attached to the second layer. The valve may be a flap integral to the second layer and formed by a slit. A stop may be configured to limit distal movement of the valve. The second layer may have a first perimeter larger than a second perimeter of the second layer. An adhesive may be on a proximal surface of the second layer. The adhesive may adhere the first layer to the second layer. The adhesive may be on a peripheral portion of the second layer to adhere the second layer to the subject. The adhesive may comprise silicon. The wound dressing may include a non-adhesive insert positioned between the adhesive and the first layer, wherein the non-adhesive insert is configured to facilitate release of the second layer from the first layer. The wound dressing may comprise an injection port attached to a distal surface of the second layer, wherein the injection port is configured to introduce the biomaterial through the first opening and the second opening. The injection port may be attached to the distal surface of the second layer with at least one adhesive. The at least one adhesive may include a first adhesive and a second adhesive, where the first adhesive is at least partially around the second opening, and the first adhesive is spaced apart from the second adhesive. The injection port may have a tab, and the second adhesive may adhere the tab to the second layer. The tab may be configured to allow a user to peel the injection port from the distal surface of the second layer. The tab may include a projection having an inner surface spaced apart from the distal surface of the second layer to allow the user to lift the tab from the distal surface of the second layer. The inner surface of the projection may be disposed at an acute angle with respect to the distal surface of the first layer. The at least one adhesive may be configured to releasably attach the injection port to the distal surface of the second layer. The at least one adhesive may have a distal surface configured to attach to the injection port and a proximal surface configured to attach to the distal surface of the second layer, and the distal surface of the at least one adhesive may have a greater tackiness than the proximal surface of the at least one adhesive to allow for release of the at least one adhesive from the distal surface of the second layer with the injection port. A kit may include the wound dressing; and a container configured to form or inject the biomaterial. The biomaterial may be a platelet rich plasma (PRP) or a platelet rich fibrin matrix (PRFM). The container may be a syringe configured to inject the biomaterial. The container may be a centrifuge tube including at least one separator gel configured to form the biomaterial.

A second aspect of the present disclosure is directed to a wound dressing for applying a biomaterial to a subject, the wound dressing comprising: a first layer comprising a first opening and a plurality of first pores having a first width; a second layer comprising a second opening, one or more second pores having a second width, and a flap configured to pivot relative to the second opening; and an injection port releasably attached to a distal surface of the second layer with a first adhesive and a second adhesive, wherein the first adhesive and the second adhesive are spaced apart, and the injection port is configured to introduce the biomaterial through the first opening and the second opening, wherein the first layer is configured to be between the second layer and the subject when the wound dressing is applied to the subject, the first opening is at least partially aligned with the second opening, the flap provides one-way passage of the biomaterial through the second opening, the second width is larger than the first width, and the one or more second pores are uncovered distally and directly open to the atmosphere.

In some embodiments, the wound dressing of the second aspect may include one or more of the following features. Each of the first adhesive and the second adhesive may have a distal surface configured to attach to the injection port and a proximal surface configured to attach to the distal surface of the second layer, and the distal surface may have a greater tackiness than the proximal surface to allow for release from the distal surface of the second layer with the injection port. The first adhesive may be at least partially around the second opening. The injection port may have a tab, and the second adhesive may attach the tab to the second layer. The tab may be configured to allow a user to peel the injection port from the distal surface of the second layer. The tab may comprise a projection having an inner surface spaced apart from the distal surface of the second layer to allow a user to lift the tab from the distal surface of the second layer. A kit may include the wound dressing; and a container configured to form or inject the biomaterial. The biomaterial comprises PRP or PRFM. The container comprises a syringe configured to inject the biomaterial. The container may be a centrifuge tube including at least one separator gel configured to form the biomaterial.

A third aspect of the present disclosure is directed to a method comprising: applying a wound dressing at least partially over a wound; and injecting a biomaterial through a first opening of a first layer of the wound dressing and a second opening of a second layer of the wound dressing to the wound, wherein air is vented through a plurality of first pores of the first layer and one or more second pores of the second layer.

In some embodiments, the method may include one or more of the following features. The injecting may be through a one-way valve of the wound dressing. The injecting may be through an injection port attached to a distal surface of the second layer. The method may further include removing the injection port from the distal surface of the second layer after injecting the biomaterial. The removing the injection port may include lifting a tab of the injection port. The method may include removing the second layer from the first layer after injecting the biomaterial. The method may include coagulating the biomaterial. The biomaterial may be platelet rich plasma (PRP) or platelet rich fibrin matrix (PRFM). The air may be vented from the second pores directly to the atmosphere.

### DEFINITIONS

To facilitate an understanding of this disclosure, a number of terms are defined below. Terms defined herein have meanings as commonly understood by a person of ordinary skill in the areas relevant to the disclosure. Terms such as “a,” “an,” and “the” are not intended to refer to only a singular entity; but include the general class of which a specific example may be used for illustration. The terminology herein is used to describe specific embodiments of the disclosure, but their usage does not limit the disclosure, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “proximal” refers to a portion or a component closer to the subject when used as intended. Conversely, the term “distal” refers to a portion or a component further from the subject when used as intended.

“Platelet poor plasma” or “PPP” is a blood-derived product from which blood cells (e.g., platelets and red blood cells) have been substantially removed. Greater than 95% RBCs may be removed from the PPP. The PRP may include less than about 0.5% residual hematocrit (e.g., less than about 0.4% residual hematocrit or less than about 0.3% residual hematocrit). The PPP may be separated from a blood sample through centrifugation in a tube with one or more separator gels that separates blood components based on density.

“Platelet rich plasma” or “PRP” is a blood-derived product when platelets are suspended in PPP. The platelets may be resuspended in the tube after the centrifugation. Due to the presence of the platelets, PRP may be stimulated to release growth factors and other proteins that promote healing. The PRP may have a liquid state and contain little to no fibrin, providing a rapid, short-term release of growth factors and other proteins. The viscosity of the PRP may be from about 1 cP to about 1.3 cP. The PRP may include at least about 2.5 x 105 platelets/µL (e.g., at least 3 x 105 platelets/µL, at least 3.5 x 105 platelets/µL, at least 4 x 105 platelets/µL, at least 4.5 x 105 platelets/µL, or at least 5.5 x 105 platelets/µL).

“Platelet rich fibrin matrix” or “PRFM” is a blood-derived product when PRP is activated to initiate a coagulation cascade, converting fibrinogen to fibrin. The PRFM may be activated by the addition of a coagulating agent such as thrombin or a calcium salt (e.g., calcium gluconate or calcium chloride). The formation of fibrin may lead to the development of a fibrin matrix stabilizer, which suspends the platelets, released growth factors, and other proteins that promote healing, allowing for a slower, sustained release of these bioactive molecules over the span of several days (e.g., from 1 day to a week, or time points therebetween). The fibrin matrix may also form a gel-like matrix. As such, the PRFM may be used for localized treatments, particularly those needing a composition having a structure (e.g., gel-like matrix). The PRFM may have various degrees of coagulation, including PRFM in a fluid state and/or in a gel state. The viscosity of the PRFM may be about 1 cP to about 1000 cP and may be dependent on the degree of fibrin polymerization and platelet concentration. The PRFM may not be fully coagulated to a gel state when removed from the tube. Accordingly, the PRFM may have a viscous, fluid state that is still capable of being administered or applied. For example, the PRFM may be removed from the tube in a liquid state having a viscosity less than 100 cP. In some embodiments, the PRFM may be at least partially coagulated in the tube to be more viscous when administered or applied. For example, the PRFM may at least partially be in a gel state when removed. The PRFM may be removed from the tube having a viscosity greater than 100 cP. Once fully formed to a gel-like state (e.g, at the wound site), the PRFM may have a viscosity of up to 1000 cP.

By “treating” or “treatment” is meant the medical management of a subject (e.g., a human) with the intent that an amelioration, repair, or prevention of an injury or disease, pathological condition, or disorder will result. This term includes active treatment, that is, treatment directed specifically toward improvement of the injury or disease, pathological condition, or disorder, and also includes causal treatment, that is, treatment directed toward removal of the cause of the injury or disease, pathological condition, or disorder. In addition, this term includes palliative treatment, that is, treatment designed for the relief of symptoms rather than the curing of the injury or disease, pathological condition, or disorder; preventive treatment, that is, treatment directed to prevention of the injury or disease, pathological condition, or disorder; and supportive treatment, that is, treatment employed to supplement another specific therapy directed toward the improvement of the injury or disease, pathological condition, or disorder.

### BRIEF DESCRIPTION OF THE DRAWINGS

The following detailed description of the embodiments of the disclosure may be better understood when read in conjunction with the appended drawings. It should be understood, however, that the disclosure is not limited to the precise arrangement and instrumentalities of the embodiments shown in the drawings.

FIG. 1 illustrates a distal view of a first embodiment of a wound dressing of the present disclosure.

FIG. 2 illustrates a side view of the wound dressing of FIG. 1.

FIG. 3 illustrates an exploded perspective view of the wound dressing of FIGS. 1 and 2.

FIG. 4 illustrates an exploded side view of the wound dressing of FIGS. 1-3.

FIG. 5 illustrates a first proximal view of the wound dressing of FIGS. 1-4.

FIG. 6 illustrates a second proximal view of the wound dressing of FIGS. 1-5 with a liner removed.

FIG. 7 illustrates an exploded view of an injection port of the wound dressing of FIGS. 1-6.

FIG. 8 illustrates a cross-sectional view of a first connector of the injection port of FIG. 7.

FIG. 9 illustrates a perspective view of the connector of FIG. 8.

FIG. 10 illustrates a distal view of the connector of FIGS. 7-9.

FIG. 11 illustrates a second side view of the wound dressing of FIGS. 1-10.

FIG. 12 is a flow chart of a method of using the wound dressing of FIGS. 1-12

FIG. 13 illustrates an exploded perspective view of a second embodiment of a wound dressing.

FIG. 14 illustrates an exploded perspective view of the wound dressing of FIG. 13.

FIG. 15 illustrates a proximal view of the wound dressing of FIGS. 13 and 14 with a releasable liner removed.

FIG. 16 illustrates a distal view of a third embodiment of a wound dressing of the present disclosure.

FIG. 17 illustrates an exploded side view of the wound dressing of FIG. 16.

FIG. 18 illustrates a first perspective view of a fourth embodiment of a wound dressing of the present disclosure

FIG. 19 illustrates a second perspective view of the wound dressing of FIG. 18.

FIG. 20 illustrates a perspective cutaway view of the wound dressing of FIGS. 18 and 19.

FIG. 21 illustrates a perspective view of a fifth embodiment of a wound dressing of the present disclosure.

FIG. 22 illustrates a perspective view of a sixth embodiment of a wound dressing of the present disclosure.

FIG. 23 illustrates a perspective view of the wound dressing of FIG. 22 with a first container and a second container attached.

FIG. 24 illustrates a perspective view of a seventh embodiment of a wound dressing according to the present disclosure.

FIG. 25 illustrates a disassembled view of the wound dressing of FIG. 24.

FIG. 26 illustrates a second perspective view of the wound dressing of FIGS. 24 and 25.

FIG. 27 illustrates a perspective view of an eighth embodiment of a wound dressing of the present disclosure.

FIG. 28 illustrates a schematic of the wound dressing of FIG. 27.

### DETAILED DESCRIPTION

The present disclosure features a device for dressing wounds. The device includes a wound dressing that may be useful for treating a wound (e.g., a burn wound, a traumatic wound, and/or a surgical wound) or an ulcer (e.g., a diabetic foot ulcer) by applying at least one of the devices described herein, e.g., using the method and kits described herein. The devices, methods, and kits can be used to deliver a biomaterial onto the skin and/or treatment site (e.g., wound or ulcer). The biomaterial may include PRP, PRFM, a coagulating agent, an antibiotic, an anesthetic, hyaluronic acid, a hydrogel, and/or an irrigation solution. The wound dressing can be used to contain the biomaterial and prevent exposure from the outside environment. Further, the wound dressing may provide ventilation of the biomaterial to the outside, atmospheric environment. The wound dressing can be used to promote the gelation (or coagulation) of the biomaterial (e.g., PRP and/or PRFM) and/or serve as a mold. As such, the devices and methods herein described can be used to provide uniform delivery of the biomaterial over the treatment site to produce a conformable gel and/or to enhance the distribution and delivery of biologics (e.g., antibiotics, therapeutics, or other drugs) within the biomaterial to the treatment site in a controlled manner.

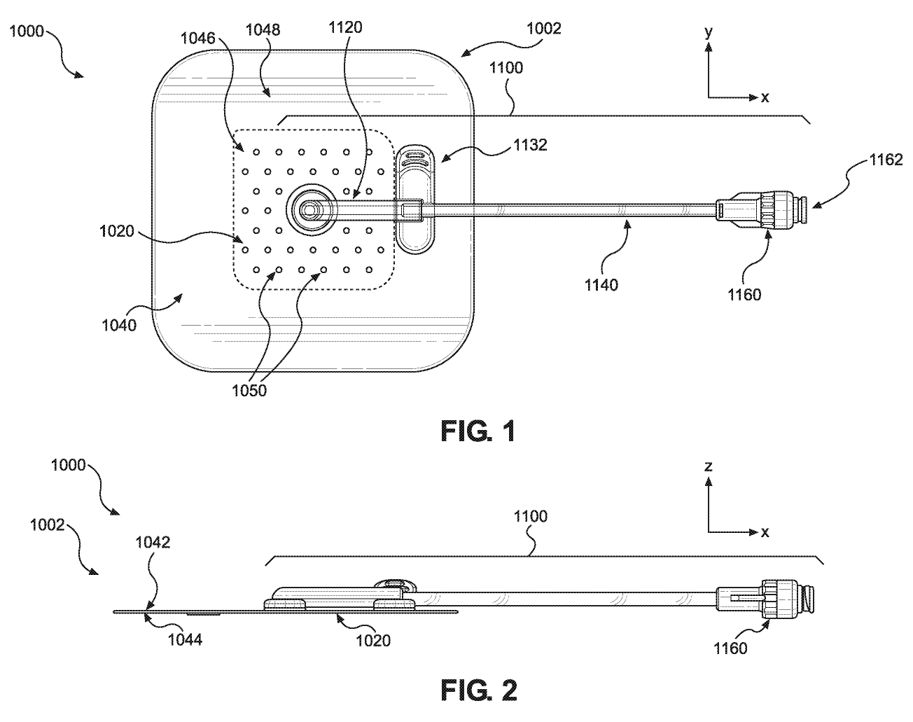

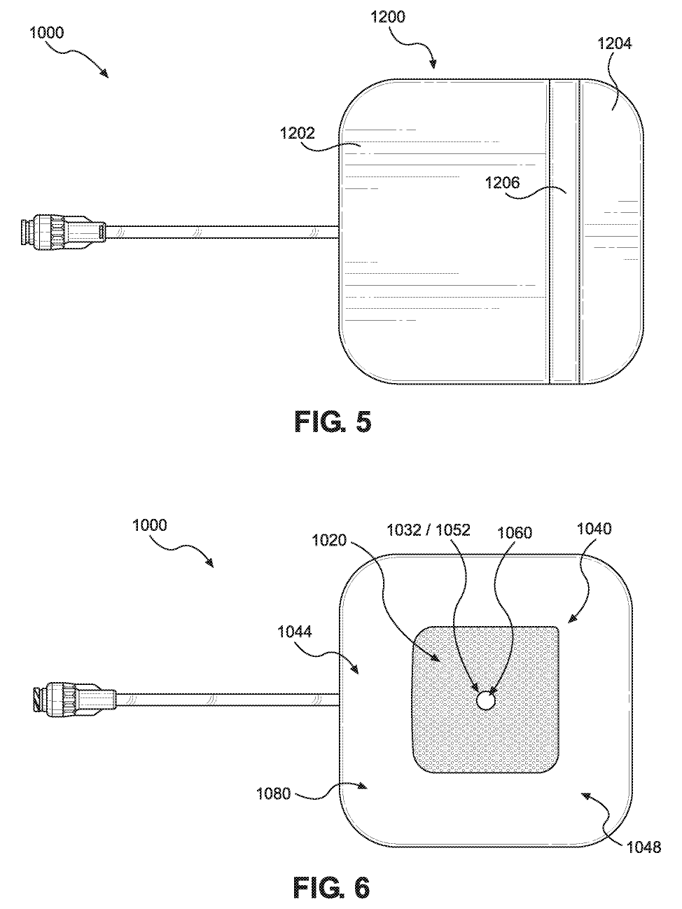

FIGS. 1-11 illustrate various views of a wound dressing 1000 of the present disclosure. As illustrated, the wound dressing 1000 may include a bandage 1002. The bandage 1002 may be applied to a subject (e.g., to skin) to retain the biomaterial in place against a wound while allowing gas (e.g., air) to pass therethrough. In some embodiments, an injection port 1100 may be attached to a distal surface of the bandage 1002 and be configured to allow passage of the biomaterial through the bandage 1002. The bandage 1002 may be flexible and/or conformable. In some embodiments, the bandage 1002 may be configured to retain a shape when bent. The bandage 1002 may conform to the application site of the subject, inhibiting leakage, and/or promoting wearability and comfort.

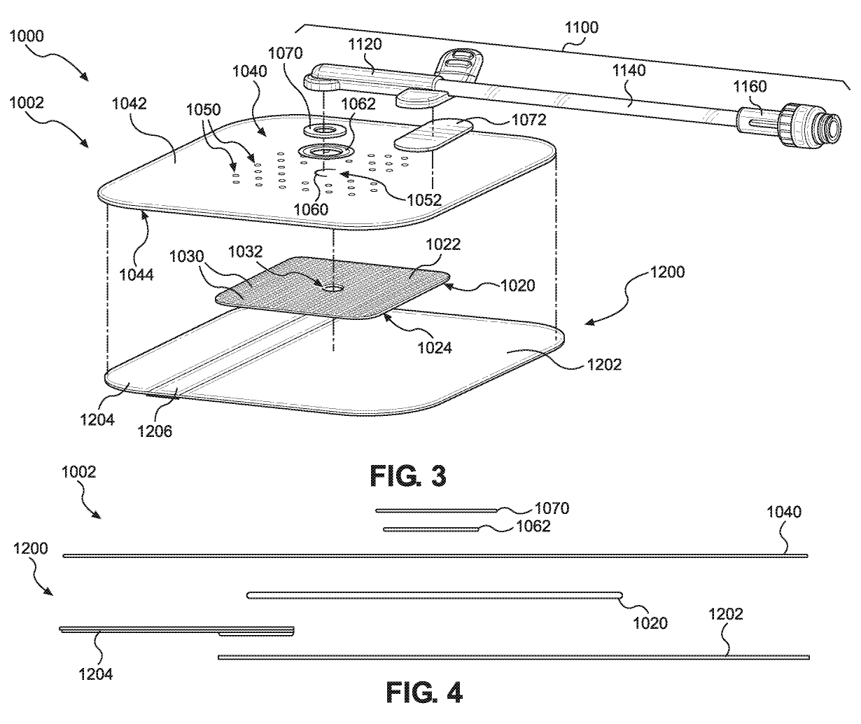

As illustrated in FIG. 3, the bandage 1002 may include a first layer 1020 and a second layer 1040. The first layer 1020 may have a distal surface 1022 and a proximal surface 1024, and the second layer 1040 may have a distal surface 1042 and a proximal surface 1044. The second layer 1040 may at least partially (e.g., fully) cover the first layer 1020, such that the first layer 1020 may be positioned between the proximal surface 1044 of the second layer 1040 and the skin of the subject. The proximal surface 1024 of the first layer 1020 may be at least partially positioned against the wound, and/or the distal surface 1042 of the second layer 1040 may be at least partially uncovered and directly open or exposed to the atmospheric environment.

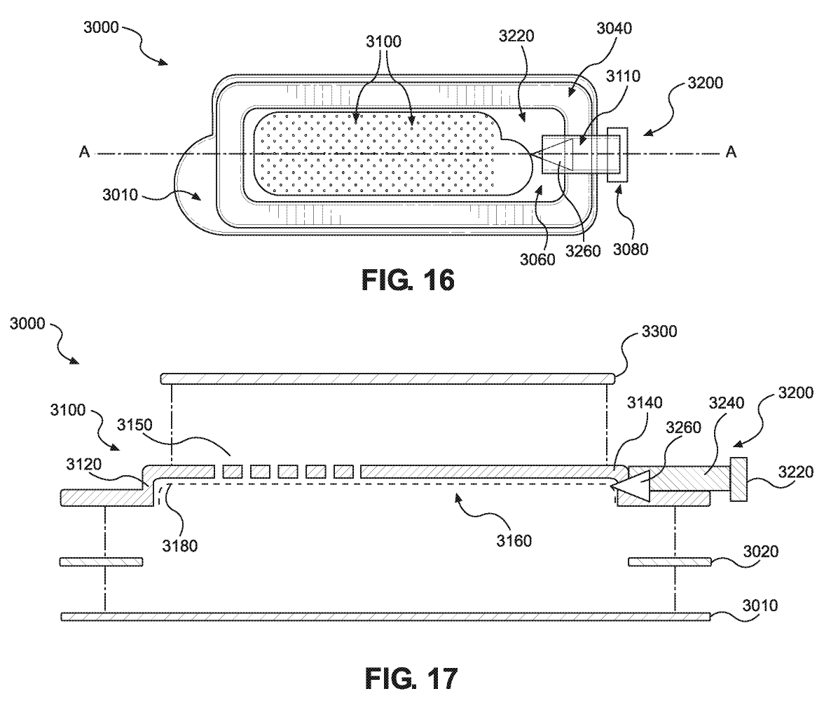

In some embodiments, the second layer 1040 may be a substantially planar sheet, as illustrated in FIG. 3. In some embodiments, the second layer 1040 may be contoured to provide a cavity, for example as illustrated in FIGS. 16 and 17.

As further illustrated in FIG. 3, the first layer 1020 may be a porous material, including a plurality of first pores 1030. The first layer 1020 may be a single layer. The first layer 1020 may be a mesh defining the first pores 1030. The first layer 1020 may be non-woven and/or non-absorbent to prevent retention of biological fluids and/or clogging of the first pores 1030. The mesh may be made polyethylene (PE), polypropylene (PP), polyester (PET), nylon (polyamide), polytetrafluoroethylene (PTFE), high-density polyethylene (HDPE), and/or polyvinylidene fluoride (PVDF).

The first pores 1030 may be sized for venting gas (e.g., air) from the area proximal of the wound dressing 1000. For example, the first pores 1030 may vent gas during injection to release gas displaced by the biomaterial directly to the atmosphere. The first pores 1030 may also allow venting once the biomaterial has been delivered to promote the drying and/or transitioning of the biomaterial to a gel state, thereby forming a gel on the skin of the subject. In some embodiments, the first pores 1030 may be liquid permeable (e.g., blood or wound exudate) but prevent passage of the biomaterial to retain the biomaterial against the wound. The first pores 1030 may have a first width of from about 0.2 microns to about 500 microns, inclusive. In some embodiments, the first width may be from about 50 microns to about 400 microns, inclusive. In some embodiments, the first width may be from about 100 microns to about 400 microns, inclusive. In some embodiments, the first width may be from about 100 microns to about 300 microns, inclusive. In some embodiments, the first width may be from about 100 microns to about 200 microns, inclusive. In some embodiments, the first width may be about 150 microns. The first layer 1020 may have at least 100 of the first pores 1030. In some embodiments, the first layer 1020 may have at least 1000 of the first pores 1030. The first pores 1030 may extend through substantially the entire surface area of the first layer 1020 forming the mesh.

The second layer 1040 may include one or more (e.g., a plurality of) second pores 1050. The second layer 1040 may be non-woven and/or non-absorbent to prevent retention of biological fluids and/or clogging of the second pores 1050. The second layer 1040 may be made of polyvinyl chloride (PVC), polyethylene (PE), and/or thermoplastic polyurethane (TPU). The second layer 1040 may be a sheet (e.g., a plastic sheet) or film (e.g., plastic film) sized to cover (e.g., fully or partially cover) the first layer 1020. The second layer 1040 may be an external cover or surface of the bandage 1002 that is at least partially uncovered by any another layer. The second pores 1050 may be directly open or exposed to the atmospheric environment, for example, without any suction or negative pressure being applied against the second layer 1040.

The second pores 1050 may be sized for venting gas (e.g., air), biomaterial (e.g., PRP or PRFM), and/or a biological fluid (e.g., blood or wound exudate) from proximal of the wound dressing 1000 to the atmosphere (e.g., at atmospheric pressure). In some embodiments, the second pores 1050 may have a shape selected from a circle, a square, a star, an oval, a rectangle, a triangle, a polygon, or any combination thereof. The second pores 1050 may have a second width from about 1 mm to about 10 mm, inclusive. In some embodiments, the second pores 1050 may have a width from about 1 mm to about 8 mm, inclusive. In some embodiments, the second pores 1050 may have a width from about 1 mm to about 4 mm, inclusive. In some embodiments, the second pores 1050 may have a width from about 1 mm to about 2 mm, inclusive. In some embodiments, the second pores 1050 may have a width from about 2 mm to about 4 mm, inclusive. The second layer 1040 may have less than one thousand of the second pores 1050. In some embodiments, the second layer 1040 may have less than one hundred of the second pores 1050. For example, as illustrated, the second layer 1040 may have less than fifty of the second pores 1050. It is also contemplated that the one or more second pores 1050 may consist of a single enlarged opening covering at least a portion of the first layer 1020.

The wound dressing 1000 may be used at atmospheric pressure, without a source of suction or vacuum. Thus, the distal surface 1042 may be at least partially uncovered and exposed to the atmospheric environment. For example, the distal surface 1042 may be uncovered except for a portion covered by the injection port 1100. The second pores 1050 may be uncovered distally with direct exposure to atmospheric air to allow the wound dressing 1000 to vent gases (e.g., air) from the wound site through the first layer 1020 and the second layer 1040, and directly to the atmospheric environment without any source of suction or vacuum. Thus, the wound dressing 1000 may allow for pressure equalization during injection of the biomaterial and promote coagulation of the biomaterial over an extended period of time without the inconvenience of external equipment.

The second layer 1040 may retain the first layer 1020 against the wound while retaining porosity of the first layer 1020. The second layer 1040 may have a second perimeter that is larger than a first perimeter of the first layer 1020. The second layer 1040 may have a second surface area that is larger than a first surface area of the first layer 1020. The second pores 1050 may be on a central portion 1046 of the second layer 1040 overlying the first layer 1020. The central portion 1046 of the second layer 1040 overlying the first layer 1020 may have a second porosity greater than a first porosity of the first layer 1020. The second width of the second pores 1050 may be greater than the first width of the first pores 1030. The first pores 1030 may be more densely arranged than the second pores. The number of first pores 1030 in the first layer 1020 may be larger than the number of second pores 1050 in the second layer 1040.

As illustrated in FIG. 1, the central portion 1046 of the second layer 1040 overlying the first layer 1020 may be centrally disposed along the surface area of the second layer 1040 (e.g., centrally disposed along an X-axis and/or a Y-axis of the second layer 1040). A perimeter portion 1048 may have a width extending at least partially around the central portion 1046. For example, as illustrated, the central portion 1046 may be centrally disposed along the X-axis and the Y-axis, forming a perimeter portion 1048 around the entire perimeter of the second layer 1040. The injection port 1110 may be adhered to the distal surface 1042 of the perimeter portion 1048.

An adhesive 1080 may be applied on the proximal surface 1044 of the perimeter portion 1048. The adhesive 1080 on the perimeter portion 1048 may adhere the bandage 1002 against the subject (e.g., the skin). In some embodiments, the adhesive 1080 may be applied to the entire proximal surface 1044 of the second layer 1040. When applied to the entire proximal surface 1044, the adhesive 1080 may adhere the first layer 1020 to the second layer 1040. The adhesive 1080 may adhere the first layer 1020 to the second layer 1040, for example by penetrating the first pores 1030. At least some of the porosity of the first layer 1020 may be retained by at least a subset of the first pores 1030 that underly the second pores 1050. The second pores 1050 may not retain the adhesive 1080 (due to the lack of a surface), thus the adhesive 1080 would not penetrate the subset of first pores 1030 underlying the second pores 1050. Thus, the second pores 1050 may be at least partially aligned with the subset of first pores 1030 to allow for ventilation through the first layer 1020 and the second layer 1040. The adhesive 1080 may comprise a low-tack adhesive to enable removal of the second layer 1040 from the subject. The adhesive 1080 may comprise a silicon adhesive. In some embodiments, the adhesive 1080 may be a silicon gel adhesive. The second layer 1040 may include a removable tab (not shown) configured to be peeled for removal of the bandage 1002.

In some embodiments, the adhesive 1080 may be applied only on the proximal surface 1044 of the perimeter portion 1048 to be applied to the subject. A second adhesive may adhere the first layer 1020 to the second layer 1040. The second adhesive (when present) may be a medical adhesive, such as an epoxy or an acrylic.

As illustrated in FIGS. 3-5, a liner 1200 may at least partially cover the proximal surface of the bandage 1002 before use. The liner 1200 may be a sheet (e.g., a plastic sheet) or film (e.g., plastic film) sized to cover the wound dressing 1000. When in place, the liner 1200 may cover the adhesive 1080, protect the bandage 1002, and/or prevent contamination of the first layer 1020 and/or second layer 1040. The liner 1200 may be a split liner. The liner 1200 may have a first liner portion 1202 and a second liner portion 1204 extending over the proximal surface. An overlap 1206 of the first liner portion 1202 and the second liner portion 1204 may have an adhesive to releasably secure the first liner portion 1202 and the second liner portion 1204. In some embodiments, the liner 1200 may have a full split, thereby providing complete exposure of the adhesive when removed (as illustrated in FIG. 6). In some embodiments, the liner 1200 may have a partial score, thereby providing partial exposure of the adhesive 1080. As illustrated in FIG. 6, the liner 1200 may be removed to expose the adhesive 1080 on the proximal surface 1044 of the second layer 1040, and the bandage 1002 may be affixed to the subject (e.g., over a wound to be treated).

The wound dressing 1000 may be configured to be placed on the subject (e.g., over a wound to be treated) before the biomaterial is applied. The bandage 1002 may have one or more openings 1032, 1052 therethrough configured to allow passage of the biomaterial. For example, the first layer 1020 may have a first opening 1032, and the second layer 1040 may have a second opening 1052. The first opening 1032 and the second opening 1052 may be at least partially aligned to allow passage of the biomaterial through the bandage 1002.

The bandage 1002 may have a valve 1060 configured to selectively provide passage of the biomaterial through the first opening 1032 and/or the second opening 1052. As illustrated in FIG. 3, the valve 1060 may be configured to extend over and close the second opening 1052. The valve 1060 may provide for one-way passage of the biomaterial through the bandage 1002 while preventing reverse passage or backflow. The valve 1060 may be self-sealing and seal upon removal of fluid pressure and/or the injection port 1100. The valve 1060 may be a flap valve, a duck valve, an umbrella valve, a resealable closure, a septum, and/or a pierceable membrane. The valve 1060 may be attached (e.g., integrally formed, welded, and/or adhered) to the first layer 1020 and/or the second layer 1040. In some embodiments, the valve 1060 may be a flap valve formed integrally from the second layer 1040. For example, the valve 1060 may be formed (at the same time as the second opening 1052) by cutting a curved slit through the second layer 1040 (such as a C-shaped slit as illustrated in FIG. 3) allowing the valve 1060 to pivot relative to the second opening 1050 (and the distal surface 1042) of the second layer 1040 to open the second opening 1050. Thus, the valve 1060 may be monolithic with the second layer 1040. The valve 1060 may be low profile requiring minimal clearance and be easy to manufacture. Additionally or alternatively, the valve (or a second valve) 1060 may be on the first layer 1020 to selectively open the first opening 1032, in a similar manner.

The valve 1060 may be configured to deflect relative to the first layer 1020 and/or the second layer 1040 to an open configuration allowing passage of the biomaterial through the first opening 1032 and/or the second opening 1052. The valve 1060 may be opened by applying fluid pressure and/or a physical force. The valve 1060 may be configured to deflect or pivot proximally to allow the biomaterial to pass through the first opening 1032 and/or the second opening 1052. Additionally or alternatively, the valve 1060 may be configured to open with physical pressure applied by a solid object, such as a needle or cannula of a syringe and/or a protrusion (e.g., 1121) of the injection port 1100. In some embodiments, the valve 1060 may be configured to be punctured and/or penetrated to be opened and self-seal to close.

As further illustrated, the valve 1060 may be configured to deflect distally to close the second opening 1052 preventing backflow of the biomaterial. The valve 1060 may be biased to revert back to the closed configuration to prevent backflow of the biomaterial through the first opening 1032 and/or the second opening 1052. For example, the natural resiliency of the material of the second layer 1040 may revert the valve 1060 distally to the closed configuration. A stop 1062 may be attached to the second layer 1040 to make the valve 1060 one-way. The stop 1062 may be attached (e.g., welded, adhered, and/or integrally formed) to the distal surface 1042 of the second layer 1040 and overlap with the valve 1060 to block further distal movement or pivoting of the valve 1060 past the distal surface 1042. The stop 1062 may be a collar that extends at least partially around the opening 1052 to contact the valve 1060.

The biomaterial may be viscous, gelled, and/or solidified when injected. The first opening 1032 and the second opening 1052 may have a width larger than at least the first pores 1030 to allow passage of the biomaterial through the first opening 1032 and the second opening 1052, while the biomaterial is prevented from passing through the first pores 1030. In some embodiments, the first opening 1032 and/or the second opening 1052 may have a width larger than the second pores 1050. The first opening 1032 may have a width greater than a width of the second opening 1052 and/or the valve 1060 to provide clearance to allow the valve 1060 to pivot proximally and open the second opening 1052. In some embodiments, the second opening 1052 and/or valve 1060 may have a width from about 1 mm to about 6 mm, inclusive. In some embodiments, the first opening 1032 and the valve 1060 may be about 3 mm. The first opening 1032 may have a width greater than the width of the second opening 1052 and/or valve 1060 to give the valve 1060 clearance to pivot proximally. The stop 1062 may have an opening with a width slightly smaller than the width of the second opening 1052 and/or valve 1060 to block the valve 1060 from pivoting distally (e.g., past the distal surface 1042) and to prevent backflow.

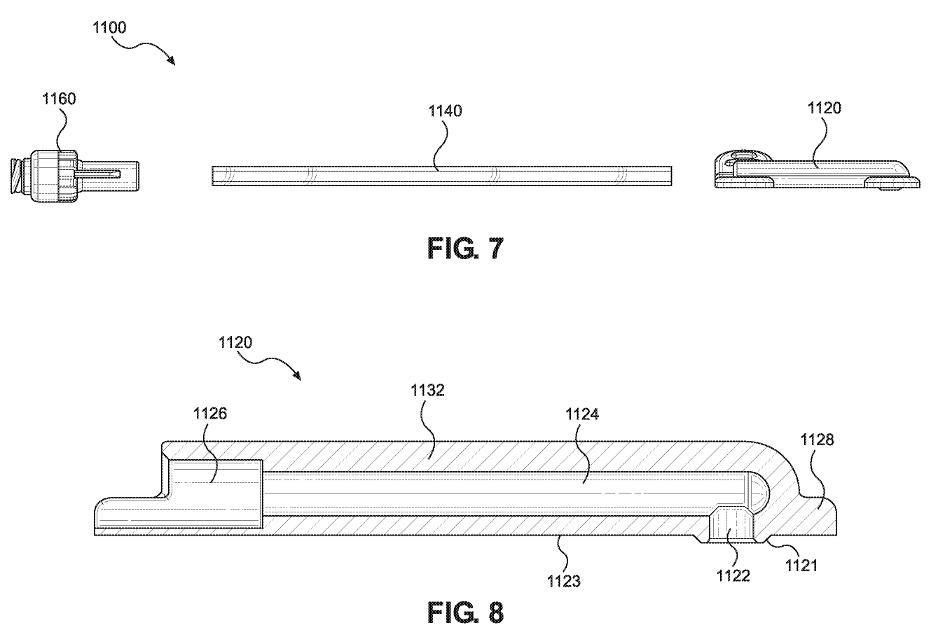

The injection port 1100 may be attached to the distal surface 1042 of the second layer 1040. The injection port 1100 may be configured to introduce the biomaterial through the bandage 1002. As illustrated in FIG. 7, the injection port 1100 may include a first connector 1120, a tube 1140, and a second connector 1160. At least a portion of the injection port 1100 may be removable from the bandage 1002 after the biomaterial is introduced into the wound. In some embodiments, a portion of the injection port 1100 may be removed. For example, the tube 1140 may be releasably attached to the first connector 1120 for the tube 1140 to be removed while the first connector 1120 remains on the bandage 1002. In some embodiments, the injection port 1100 may be removed from the bandage 1002 in its entirety to increase comfort and convenience to the subject after the biomaterial is injected.

As illustrated in FIG. 8, the first connector 1120 may have an outlet 1122 at a first end portion. The outlet 1122 may be aligned with the first opening 1032 and/or the second opening 1052 for passage of the biomaterial through the bandage 1002. In some embodiments, the first connector 1120 may extend through the first opening 1032 and/or the second opening 1052 to prop open the valve 1060 of the bandage 1002 when in place and close the valve 1060 when the first connector 1120 is removed from the second layer 1040. For example, the first connector 1120 may have a protrusion 1121 extending proximally from and/or past a proximal surface 123 of the first connector 1120. The protrusion 1121 may extend at least partially (e.g., entirely) around the outlet 1122. In some embodiments, the protrusion 1121 may prop open the valve 1060 when positioned on the second layer 1040 to at least partially open the second opening 1052. In some embodiments, the first connector 1120 (e.g, the protrusion 1121) may not physically prop open the valve 1060 and be configured to open the valve 1060 of the bandage 1002 with fluid pressure.

As further illustrated in FIG. 8, the first connector 1120 may include a channel 1124 in fluid communication with the outlet 1122. The channel 1124 may extend along the distal surface 1042 of the second layer 1040. The outlet 1112 may extend substantially perpendicular to the channel 1124 (e.g., at a 90° bend) to direct the biomaterial from the channel 1124 through the bandage 1002. The first connector 1120 may include a valve (not shown) in the outlet 1122 and/or the channel 1124 configured to prevent backflow of the biomaterial and/or provide redundancy to the valve 1060.

As illustrated in FIG. 7, the tube 1140 may connect the first connector 1120 and the second connector 1160. The tube 1140 may spatially separate the second connector 1160 from the first connector 1120 to facilitate handling and injection of the biomaterial without disturbing the bandage 1102. The tube 1140 may be flexible to prevent forces to be applied to the bandage 1102 when a container (e.g., a syringe) of the biomaterial is attached to the second connector 1160. The tube 1140 may be received in a counterbore 1126 at a second end portion of the first connector 1120. The tube 1140 may be permanently attached in the counterbore 1126 when the injection port 1100 is configured to be removed in its entirety. In some embodiments, the tube 1140 may be releasably retained in the counterbore 1126 when the tube is removed separately from the first connector 1120. The tube 1140 may be longitudinally aligned with the channel 1124 of the first connector 1120 and a channel (not shown) of the second channel 1160.

The second connector 1160 may provide access for the biomaterial to be passed through the tube 1140, the first connector 1120, and the bandage 1002. The second connector 1160 may receive an end of the tube 1140 in a first end portion and have an inlet port 1162 on a second end portion configured to connect to the container of the biomaterial. The inlet port 1162 may have an opening and a connection, such as a Luer fitting (e.g., Luer slip or Luer lock), to fluidly couple to the container without leakage. The inlet port 1162 may further include an internal valve (not shown, e.g., a Luer-activated valve) configured to inhibit backflow of the biomaterial out of the inlet port 1162. The inlet port 1162 may be configured to provide access through the bandage 1002 after application of the wound dressing 1000 to the subject. The inlet port 1162 may have an opening with a width from about 0.5 mm to about 10 mm.

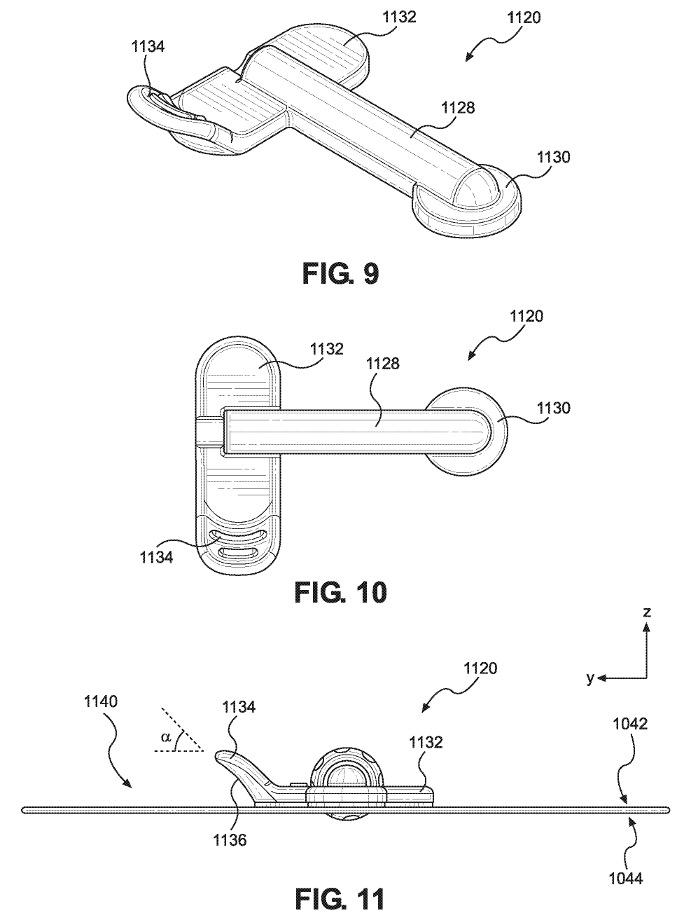

As further illustrated in FIGS. 9-11, the first connector 1120 may have a tubular portion 1128 defining the channel 1124, a flange 1130 at a first end portion of the tubular portion 1128, and a tab 1132 at a second end portion of the tubular portion 1128. The first connector 1120 may have an elbow (e.g., a 90° bend) proximate the outlet 1122 to direct the flow from the tubular portion 1128 through the outlet 1122 and the bandage 1002. The flange 1130 may extend at least partially around the outlet 1122 to attach the first connector 1120 to the bandage 1002.

The injection port 1100 may be attached to the bandage 1002 with at least one adhesive. For example, the injection port 1100 may be (directly or indirectly) connected to the distal surface 1042 of the second layer 1040 at two or more spaced-apart locations, as illustrated in FIG. 3. The flange 1130 may be attached to the distal surface 1142 of the second layer 1040 with a first adhesive 1070. The first adhesive 1070 may be substantially annular (e.g., a washer) and extend at least partially (e.g., entirely) around the opening 1152 of the second layer 1040. The first adhesive 1070 may be applied against the proximal surface of the flange 1130, and the protrusion 1121 may extend the thickness of the first adhesive 1070 to prevent the first adhesive 1070 from contacting the biomaterial passing through the outlet 1122 to avoid wetting. The first adhesive 1070 may be (directly or indirectly) adhered to the distal surface 1042 of the second layer 1040. For example, the first adhesive 1070 may be adhered to the stop 1062 that is secured to the distal surface 1042 of the second layer 1040

The tab 1132 may have an elongated shape and extend substantially perpendicular to the tubular portion 1128. The tab 1132 may be (directly or indirectly) attached to the distal surface 1142 of the second layer 1040 with a second adhesive 1072. As further illustrated in FIGS. 9-11, the tab 1132 may extend across the tubular portion 1128 to provide a secure attachment on both sides of the tubular portion 1128. The second adhesive 1072 may have an elongated, tab shape to attach the tab 1132 to the second layer 1040. The first adhesive 1070 and the second adhesive 1072 may be spaced apart along the length of the tubular portion 1128. The two points of attachment may reduce strain on the attachment of the first connector 1120 to the second layer 1040.

The injection port 1100 may be releasably attached to the bandage 1002 to be removed from the bandage 1002 after injection of the biomaterial. The tab 1132 may have a projection 1134 configured to facilitate removal of the first connector 1120. As illustrated in FIG. 11, the projection 1134 may have an inner surface 1136 distally spaced from the distal surface 1042 of the second layer 1040. The inner surface 1136 may allow a user to lift, pry, or peel the tab 1132 from the distal surface 1042 of the second layer 1040 to remove the first connector 1120 and the injection port 1100 from the bandage 1102. The inner surface 1136 may extend substantially at an acute angle α relative to the distal surface 1042 (e.g., y-axis) of the second layer 1040 to create a space to receive a finger of the user for the lifting, prying, or peeling motion. The acute angle α may be from about 20° to about 60° relative to the y-axis of the second layer 1140. In some embodiments, the acute angle α may be about 45° relative to the y-axis of the second layer 1140. Lifting the inner surface 1136 may be configured to allow removal of the injection port 1100 from the two or more locations due to a rigidity of the first connector 1120.

The first adhesive 1070 and the second adhesive 1072 may configured to facilitate the removal of the injection port 1100. Each of the first adhesive 1070 and the second adhesive 1072 may have a first side that has a greater tackiness or adhesiveness than a second side for a controlled release of the first connector 1120. For example, each of the first adhesive 1070 and the second adhesive 1072 may have a distal side that attaches to the first connector 1120 and a proximal side that attaches to the second layer 1040. The distal side have a greater tackiness or adhesiveness than the proximal side to ensure that the adhesive 1070, 1072 is removed from the bandage 1102 with the first connector 1120. The first adhesive 1070 and/or the second adhesive 1072 may comprise a double-sided tape that secures the first connector 1120 to the second layer 1040. The first adhesive 1070 and/or the second adhesive 1072 may be a tape formed of a hydrogel and/or silicon gel. The hydrogel and/or silicon gel may be coated onto a carrier comprising a polyurethane and/or polyester film. The first adhesive 1070 and/or the second adhesive 1072 may further have an acrylic layer and a polyester layer. The acrylic layer and the polyester layer may have different adhesive properties. The acrylic layer may have a stronger adherence than the polyester layer. The acrylic layer may be attached to a proximal surface of the injection port 1100, and the polyester layer may be attached to the distal surface 1042 of the second layer 1040.

The second layer 1040 may have a surface area of a square, circular, oval, rounded square, or rectangular shape. In some embodiments, the first layer 1020 and/or the second layer 1040 may a length, width, and/or maximum diameter of about 0.5 cm to about 100 cm. In some embodiments, the first layer 1020 may have a length of about 5 cm and a width of about 5 cm, and the second layer 1040 may have a length of about 10 cm and a width of about 10 cm. In some embodiments, the first layer 1020 may have a length of about 8 cm and a width of about 2 cm, and the second layer 1040 may have a length of about 10 cm and a width of about 10 cm. In some embodiments, the first layer 1020 may have a length of about 15 cm and a width of about 15 cm, and the second layer 1040 may have a length of about 8 cm and a width of about 2 cm.

For example, the wound dressing 1000 may have a round shape. In some embodiments, the wound dressing 1000 may have a diameter of about 3 cm. In another example, the wound dressing 1000 may have an oval shape, a length of about 10 cm, a width of about 3 cm. In another example, the wound dressing 1000 configured to treat a linear wound may have a rectangular shape, a length of about 8 cm and a width of about 2 cm. In another example, the wound dressing 1000 configured to treat a large wound may have a square shape, a length of about 6 cm and a width of about 6 cm.

In some embodiments, the first layer 1020 may have an additive. The additive may be a coagulant, a stabilizing agent, an anticoagulant, a drying agent and/or an antibacterial agent. The additive may be configured to contact the biomaterial. For example, the additive may be a coagulating agent, such as a calcium salt, an aluminosilicate (e.g., kaolin), diatomaceous earth, a polysaccharide, and/or a polysaccharide (e.g., chitosan). The calcium salt may be calcium gluconate and/or calcium chloride. The coagulating agent may be configured to coagulate or solidify the biomaterial. The stabilizing agent may be ascorbic acid, sodium ascorbate, lactate, gluconic acid, and/or citrate. The anticoagulating agent may be buffered citrate, sodium citrate, citrate phosphate dextrose (CPD), ethylenediaminetetraacetic acid (EDTA), heparin, oxalate, anticoagulant citrate dextrose A (ACD-A), any derivatives thereof, or any combinations thereof. The anticoagulant may inhibit the coagulation of the biomaterial (e.g., PRP or PRFM) when injected. The additive may also be present in the form of a coating, a liquid, a powder, a pellet, and/or other particulates. The additive may be coated on the proximal surface 1024 of the first layer 1020. In some embodiments, the additive may be spray-coated onto the proximal surface 1024 of the first layer 1020 in a particulate form. In some embodiments, the additive may be kaolin in an amount of from about 0.5 mg to about 2000 mg.

The present disclosure also provides a kit including the wound dressing 1000. The kit may include the bandage 1002 and/or the injection port 1100 (attached together or separated), at least one sterilization, at least one needle, and/or a biomaterial dispenser. In some embodiments, the kit may include solutions and containers to form PRP and/or PRFM. For example, the kit may include a centrifuge tube including one or more separator gel (e.g., thixotropic) to separate a blood sample into components. In some embodiments, the centrifuge tube may be pre-filled with drug layers, such as an anticoagulant, a coagulating agent, and/or a stabilizing agent.

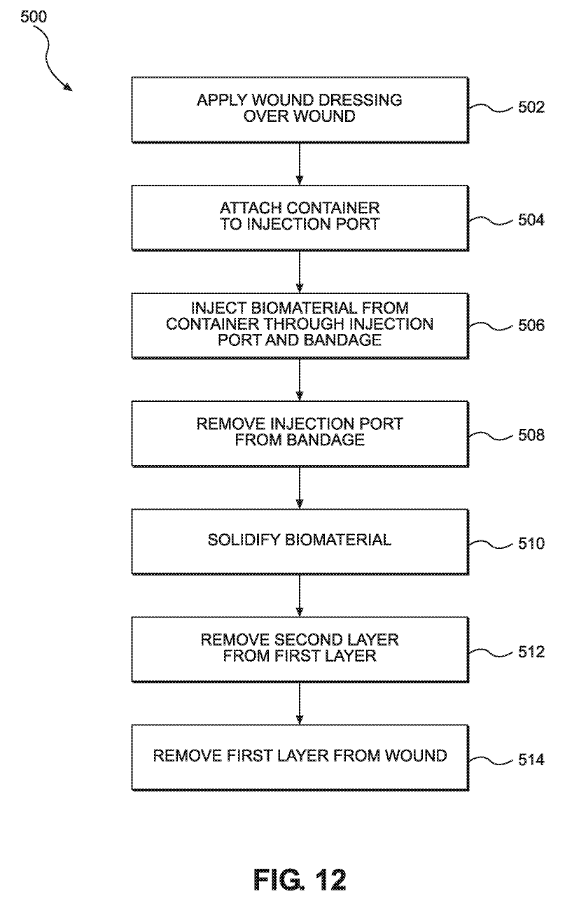

FIG. 12 illustrates a method 500 of applying a biomaterial to a wound.

In some embodiments, the biomaterial is a blood-derived product such as PRP or PRFM, and the method may initially include producing the PRP or PRFM. For example, the method 500 may include separating PPP from a blood sample through centrifugation with one or more separator gels. Platelets may then be resuspended into the PPP to form PRP. In some embodiments, the method may include activating the plasma and/or platelets to form PRFM in the centrifuge tube. The biomaterial may then be transferred to a container to be applied to the wound. The container may be a syringe, a hand pump, an electric pump, and/or a mechanical pump.

In step 502, the wound dressing 1000 may be applied to the subject (e.g., over a wound). Prior to adhering the wound dressing to the subject, the method may include sterilizing the application site (e.g., the wound site) of the subject using a sterilization solution or wipe and allowing the application site to dry. The sterilization may provide better adhesion of the wound dressing 1000 on the application site while also reducing potential contamination of the biomaterial. The wound dressing 1000 may then be removed from a packaging.

As discussed above, the liner 1200 may be removed from a proximal surface of the bandage 1002 to expose the first layer 1020 and the adhesive 1080 on the proximal surface 1044 of the second layer 1040. The bandage 1002 may then be applied over the wound. The first layer 1020 may be positioned to substantially cover the wound. The perimeter portion 1048 of the proximal surface 1044 may be positioned around the wound, and the adhesive 1080 on the perimeter portion 1048 may secure the bandage 1002 on the subject.

In step 504, the container containing the biomaterial may be attached to the injection port 1100. The container may have a chamber containing the biomaterial. The container may be configured to releasably attach to the inlet port 1162 of the second connector 1160. In some embodiments, the container may have a Luer connector configured to be attached to a luer connector on the inlet port 1162.

In step 506, the biomaterial may be injected from the container through the injection port 1100 and the bandage 1002. The biomaterial may pass through the second connector 1160, the tube 1140, and the first connector 1120. In some embodiments, the tube 1140 may be substantially rigid providing a linear passage of the biomaterial to reduce resistance of the biomaterial. In some embodiments, the tube 1140 may be flexible to facilitate attachment of the container. The first connector 1120 may guide the biomaterial with the bend proximally toward the wound. The biomaterial may push the valve 1060 open to enable passage through the second opening 1052 of the second layer 1040 and the first opening 1032 of the first layer 1020. The biomaterial may displace gas (e.g., air) that passes through the first pores 1030 and the second pores 1050 as the biomaterial is passed through bandage 1002 and into contact with the wound. The gas may be vented through the first pores 1030 and the second pores 1050. The valve 1060 may close when the biomaterial is completely passed the valve 1060 to retain the biomaterial against the wound. The biomaterial may be in a liquid state, a gel-like, gel, and/or solid state when delivered. The injection port 1100 and openings 1032, 1052 may be sized for the passage of biomaterial with various viscosities. The wound dressing 1000 may be particularly suitable for viscous, liquid biomaterials due to the larger size of the first opening 1032 and the second opening 1052, and the first pores 1030 retaining the biomaterial against the wound. For example, the biomaterial, when injected, may have a viscosity of up to 100 cP.

In step 508, the injection port 1100 may be removed from the bandage 1002. For example, the user may peel the first connector 1120 from the second layer 1040 by lifting the projection 1134 of the release tab 1132. The first adhesive 1170 and the second adhesive 1172 may detach from the second layer 1040 to be removed with the injection port 1100. The bandage 1102 may remain in place to retain the biomaterial against the wound and to cover and protect the wound. Removal of the injection port 1100 may ensure comfort of the subject as the bandage 1102 covers the wound and the wound heals.

In step 510, the biomaterial may solidify and/or coagulate and be vented through the bandage 1102. The ventilation of the bandage 1102 may facilitate the solidification and/or coagulation by providing a sufficient airflow. The time required for coagulation of the biomaterial may be about 10 minutes to about 2 hours. The biomaterial may conform to the wound as it solidifies and/or coagulates. The biomaterial (e.g., PRP and/or PRFM) may provide a fibrous scaffold rich in structural protein and growth factors improving the healing of the wound.

In step 512, the second layer 1040 may be removed from the first layer 1020. The second layer 1040 may be removed before or after the biomaterial solidifies and/or coagulates. For example, the second layer 1040 may provide ventilation to the biomaterial as it solidifies and/or coagulates, and then the second layer 1040 may be removed. The second layer 1040 may be removed about 12 hours to about 4 days after the biomaterial is applied to the wound. The second layer 1040 may be removed by peeling the second layer 1040 off of the skin and the first layer 1020, detaching the adhesive 1080 from each. The first layer 1020 may be retained after the second layer 1040 is removed to protect the wound and/or retain the biomaterial. An additional wrap or bandage may retain the first layer 1020 in place. The additional wrap or bandage may be adhesive free to prevent irritation of the skin.

In step 514, the first layer 1020 may be removed from the wound. The first layer 1020 may be removed from the subject after the wound is substantially healed. For example, the first layer 1020 may be removed about 4 days to about 14 days after the biomaterial is applied to the wound.

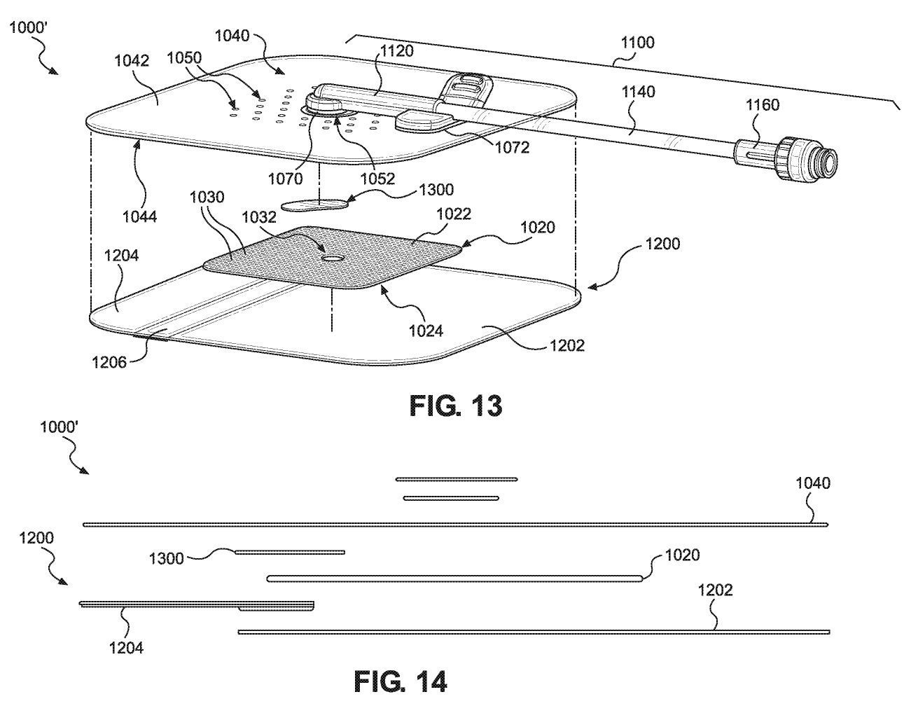

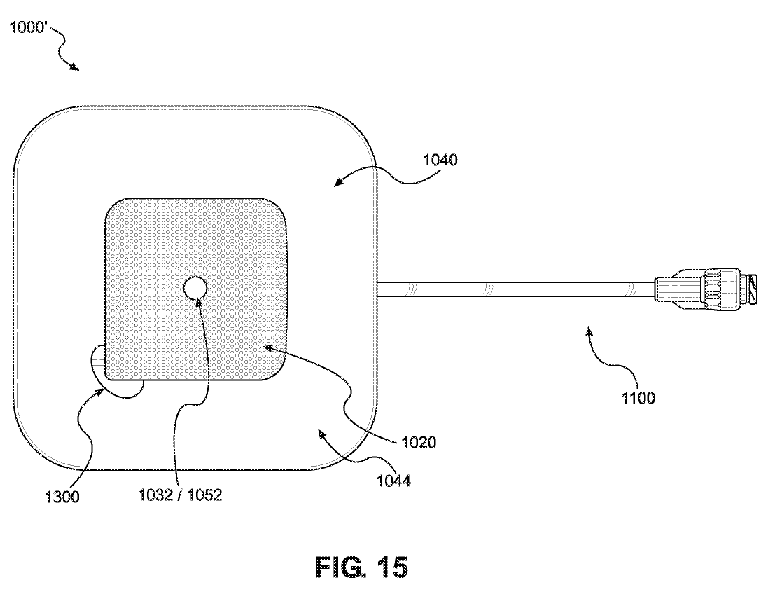

FIGS. 13-15 illustrate a second embodiment of a wound dressing 1000’. The wound dressing 1000’ may have one or more of the same features as disclosed for the wound dressing 1000, the entire disclosure of which is incorporated herein for sake of brevity. For example, the wound dressing 1000’ may have all of the features of the wound dressing 1000 as disclosed. The wound dressing 1000’ may be used in the method 500, as discussed.

As further illustrated, the wound dressing 1000’ may further include at least one non-adhesive insert 1300 configured to facilitate separation of the second layer 1040 from the first layer 1020 to allow removal of the second layer 1040 while maintaining the first layer 1020 at the wound site, for example as discussed with reference to Step 512. The non-adhesive insert 1300 may provide an area of non-adhesion to facilitate fingers grabbing a corner of the first layer 1020. The non-adhesive insert 1300 may be positioned between the proximal surface 1044 of the second layer 1040 and the distal surface 1022 of the first layer 1020 to ensure that the adhesive 1080 of the second layer 1040 does not stick to a portion of the first layer 1020. For example, as illustrated, the non-adhesive insert 1300 may positioned at a corner of the first layer 1020 to facilitate peeling of the second layer 1040 at the corner of the wound dressing 1000’. When in position, the non-adhesive insert 1300 may extend laterally from the first layer 1020 outside of the perimeter of the first layer 1020 to allow the user to wedge a finger between the non-adhesive insert 1300 and the second layer 1040. As illustrated, the non-adhesive insert 1300 may be positioned at a single corner of the first layer 1020 to provide an asymmetric peeling advantage of the second layer 1040. For example, the corner of the first layer 1020 may be indicated by the corresponding corner of the second layer 1040. The corner of the second layer 1040 may have a peel tab (not shown) indicating alignment with the corner of the first layer 1020 having the non-adhesive insert 1300. Additionally or alternatively, the corner of the second layer 1040 may be indicated by another indicator, such as a visual indicator (e.g., a marking and/or indentation) and/or tactile indicator (e.g., a textured pattern) on the distal surface 1042 of the second layer 1040. Additionally or alternatively, the wound dressing 1000’ may include a plurality of the non-adhesive inserts 1300. For example, when the first layer 1020 and/or the second layer 1040 is a quadrilateral (as shown), a plurality of the corners (e.g., each corner) of the first layer 1020 may each have a non-adhesive insert 1300.

FIGS. 16 and 17 illustrate a third embodiment of a wound dressing 3000. The wound dressing 3000 may have one or more of the same features as the previously disclosed embodiments of the wound dressing 1000, 1000’, the entire disclosures of which are incorporated herein for sake of brevity. The wound dressing 3000 may be used in the method 500, as discussed.

As illustrated, the wound dressing 3000 may include a housing 3100 and a cover 3300.

The housing 3100 may have a frame 3120 and a ceiling 3140. In some embodiments, the frame 3120 and the ceiling 3140 may be made of one-piece. In some embodiments, the frame 3120 and the ceiling 3140 may be made of two pieces fixed together. The ceiling 3140 may be a distal portion of the frame 3120. The ceiling 3140 may have a proximal surface that is configured to face the skin of the subject when the wound dressing 3000 is applied. In some embodiments, the ceiling 3140 defines a flat or contoured surface. A distal surface of the ceiling 3140 may configured to face towards the environment and away from the skin. The ceiling 3140 may include a plurality of ribs, a raised ceiling (e.g., an outward projecting dome), and/or a surface finish (e.g., pits, bumps, and/or groves (e.g. to resemble skin texture)).

The frame 3120 may extend vertically from the skin to separate the ceiling 3140 from the skin and form a cavity 3160 between the ceiling 3140 and the skin. The cavity 3160 may have a width, length, and/or a maximum diameter of about 0.5 cm to about 50 cm. The length and width of the cavity 3160 may be the same or different. The cavity 3160 may have a circular, oval, rounded square, or rectangular shape. The dimensions and/or shape of the cavity 3160 may be defined by the size, depth, volume, and/or shape of the wound. The cavity 3160 may serve as a mold for the biomaterial as it dries and/or coagulates, such that the biomaterial may be formed in the shape of the cavity 3160 upon removal of the wound dressing 3000. The biomaterial may be molded by the shape of a plurality of ribs, a raised ceiling, and/or a surface finish. The plurality of ribs may be hidden behind a reveal. The plurality of ribs may be configured to allow an expansion of the volume of the cavity 3160. For example, once the volume of biomaterial injected into the cavity 3160 has reached the minimum fill volume of the cavity 3160, the biomaterial may flow into the plurality of ribs, thereby expanding the cavity 3160. The additional volume of biomaterial may be injected to form a textured distal surface of the biomaterial, such that the texture is formed by the plurality of ribs and/or the surface finish. The raised ceiling may also allow an additional volume of biomaterial beyond that of the minimum fill volume to be injected into the cavity 3160. The biomaterial may then conform to the shape of the frame 3120 and/or the ceiling 3140. For example, the distal side of the biomaterial may form a dome shape. In some embodiments, the frame 3120 may be omitted such that any vertical frame is omitted and the ceiling 3140 is a flat sheet that lies substantially against the skin of the subject.

The wound dressing 3000 may have an injection port 3200. The injection port 3200 may have an inlet 3220 and a tube 3240. The injection port 3200 may be configured to introduce the biomaterial into the cavity 3160 through the inlet 3220 and the tube 3240. A valve 3260 configured prevent backflow of the biomaterial. The valve 3260 may be attached to the housing 3100, the inlet 3220, and/or the tube 3240. In some embodiments, the valve 3260 may be attached to the housing 3100, and the injection port 3200 may be removable after the biomaterial is introduced into the housing 3100. The injection port 3200 may have one or of the features as discussed above with respect to the wound dressing 1000, the entire disclosure of which is incorporated herein by reference.

The housing 3100 may have a plurality of first pores 3150 through the ceiling 3140, as illustrated. The first pores 3150 may be sized to allow for ventilation of the cavity 3160, such as allowing the passage of gas (e.g., air) as the biomaterial is introduced into the housing 3100. In some embodiments, the first pores 3150 may be sized to allow a fluid (e.g., an exudate) to escape from the cavity 3160 while the wound dressing 3000 is in place on the skin after removal of the cover 3300. In some embodiments, the first pores 3150 may further be sized to allow for the biomaterial to be pass therethrough, for example when excess biomaterial was injected into the cavity 3160.

The cover 3300 may be applied to the distal surface of the housing 3100. The cover 3300 may at least partially cover the first pores 3150. The cover 3300 may be releasably attached to the distal surface of the housing 3100. The cover 3300 may have a plurality of second pores (not shown). In some embodiments, the first pores 3150 may be larger than the second pores of the cover 3300. The second pores may be sized for venting gas (e.g., air) from the cavity 3160 while inhibiting the escape of biomaterial out from the cavity 3160. In some embodiments, the second pores may also be sized for the passage of a fluid (e.g., exudate) from the internal cavity. Alternatively, the second pores in the cover 3300 may be larger than the first pores 3150, as discussed above with respect to the wound dressing 1000, the entire disclosure of which is incorporated herein by reference. The second pores may be distributed across the cover 3300 in distinct central, intermediate, and peripheral zones (e.g., the central zone that overlies the first layer and internal cavity may exhibit the highest coverage of second pores). The second pores may comprise pores that may occupy various density ranges across the entirety of the cover 3300 (e.g., the central region of the cover 3300 may have more pores than the intermediate region or the peripheral region).

The cover 3300 may be releasably attached to the housing 3100, e.g., with an adhesive. The cover 3300 may be removed (e.g., peeled off) after the biomaterial is injected into the cavity 3160 to a desired volume. The cover 3300 may include a pull-tab extending outside the area of the housing 3100 and/or wound dressing 3000 to allow the cover 3300 to be peeled off. The cover 3300 may be removed once the biomaterial is coagulated or solidified after injection into cavity 3160 (e.g., a few hours (e.g., less than 24 hours) after injection of the biomaterial). The removal of the cover 3300 may expose the pores 3150.

The cover 3300 may be a plastic sheet or plastic film. The cover 3300 may be made of a polyvinyl chloride (PVC), polyethylene (PE), thermoplastic polyurethane (TPU), or any biocompatible polymer. The cover 330 may include a slit to accommodate (e.g., fit around) the injection port 3200 of the wound dressing.

The housing 3100 may include an additive 3180 on one or more of the internal walls of the cavity 3160. As discussed above, the additive may be a coagulant, a stabilizing agent, an anticoagulant, a drying agent and/or an antibacterial agent. The additive may be configured to contact the biomaterial. For example, the additive may be a coagulating agent, such as a calcium salt, an aluminosilicate (e.g., kaolin), diatomaceous earth, a polysaccharide, and/or a polysaccharide (e.g., chitosan). The calcium salt may be calcium gluconate and/or calcium chloride. The coagulating agent may be configured to coagulate or solidify the biomaterial. The stabilizing agent may be ascorbic acid, sodium ascorbate, lactate, gluconic acid, and/or citrate. The anticoagulating agent may be buffered citrate, sodium citrate, citrate phosphate dextrose (CPD), ethylenediaminetetraacetic acid (EDTA), heparin, oxalate, anticoagulant citrate dextrose A (ACD-A), any derivatives thereof, or any combinations thereof. The anticoagulating agent inhibits the coagulation of the biomaterial (e.g., PRP or PRFM) in the internal cavity. The additive may also be present in the form of a coating, a liquid, a powder, a pellet, and/or other particulates In some embodiments, the additive may be spray-coated in a particulate form onto the one or more of the internal walls of the cavity 3160.

At least a portion of the housing 3100 may be optically transparent to visualize the biomaterial as it enters the cavity 3160. Further, the housing 3100 may be flexible, deformable, and/or conformable. The ceiling 3140 may be provided in the form of a sheet, membrane, hydrogel, or a solid material. The housing 3100 may be made from a polymer, a silicon, a foam, a mesh, a hydrogel, any derivatives thereof, or any combinations thereof. The polymer and/or hydrogel may be made from or may include polyethylene, polyester, vinyl, and/or any biocompatible materials (e.g., silicone or PVC).

The housing 3100 may have a proximal contact surface coated with an adhesive 3020. The adhesive 3020 may seal the cavity 3160 from the outside environment when applied to skin. The adhesive 3020 may comprise a low-tack adhesive to enable removal of the wound dressing 3000 from the subject. The adhesive 3020 may be a silicon gel adhesive.

A liner 3010 may be configured to cover the adhesive 3020 and enclose the cavity 3160. The liner 3010 may be removed prior to use to expose the adhesive 3020 and the cavity 3160. In some embodiments, the liner 3010 may be a single sheet. In some embodiments, the liner 3010 may be a split liner, as discussed with reference to the wound dressing 1000, the entire disclosure of which is incorporated herein.

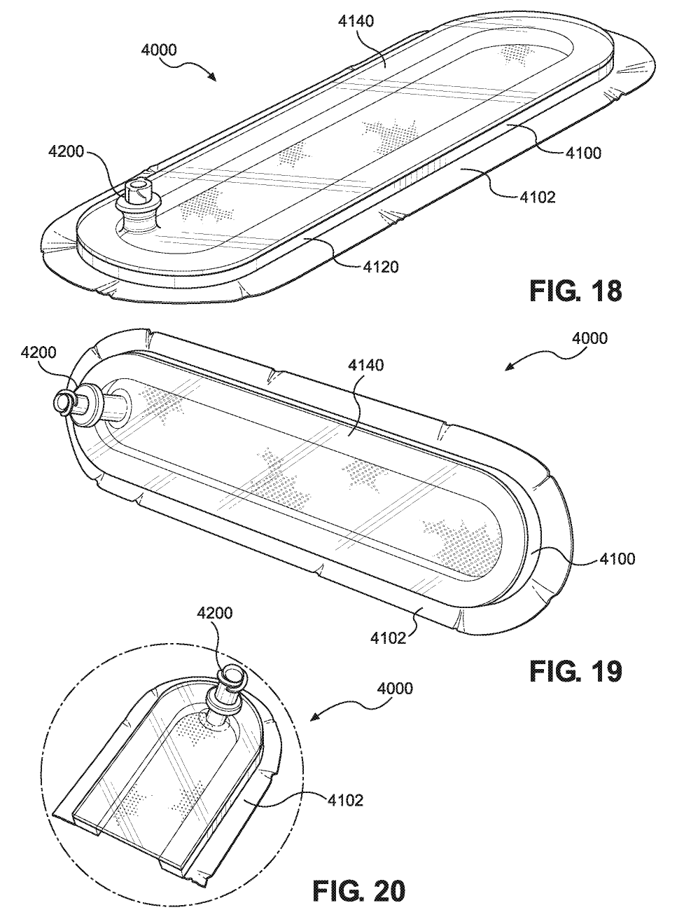

FIGS. 18-20 illustrate a fourth embodiment of a wound dressing 4000. The wound dressing 4000 may have one or more of the features as the previously disclosed embodiments of the wound dressing 1000, 1000’, and/or 3000, the entire disclosure of which is incorporated herein for sake of brevity. For example, the wound dressing 4000 may include one or more features of the wound dressing 3000. The wound dressing 4000 may be used in the method 500, as discussed. As further illustrated, the wound dressing 4000 may include a housing 4100 having a frame 4120 and a ceiling 4140. The housing 4100 may further include a skirt 4102 extending at least partially around the perimeter of the housing 4100 for increased surface contact between the wound dressing and the application site. An injection port 4200 may extend through a distal surface of the ceiling 4140. For example, the injection port 4200 may extend substantially perpendicular to a surface area of the ceiling 4140.

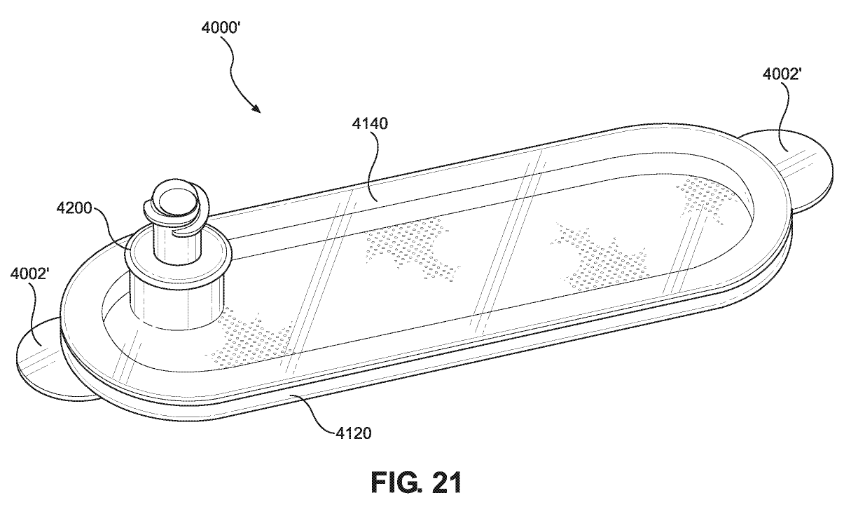

FIG. 21 illustrates a fifth embodiment of a wound dressing 4000’. The wound dressing 4000’ may have one or more of the same features as the previously disclosed embodiments of the wound dressing 1000, 1000’, 3000, and/or 4000, the entire disclosures of which is incorporated herein for sake of brevity. For example, the wound dressing 4000’ may have all of the features of the wound dressing 3000 and/or the wound dressing 4000, as disclosed. The wound dressing 4000’ may be used in the method 500, as discussed. As further illustrated, the wound dressing 4000’ may include a housing 4100 having a frame 4120 and a ceiling 4140. An injection port 4200 may extend through a distal surface of the ceiling 4140. For example, the injection port 4200 may extend substantially perpendicular to a surface area of the ceiling 4140. The wound dressing 4000’ may further include one or more pull tabs 4002’ to remove the wound dressing 4000’ from the subject.

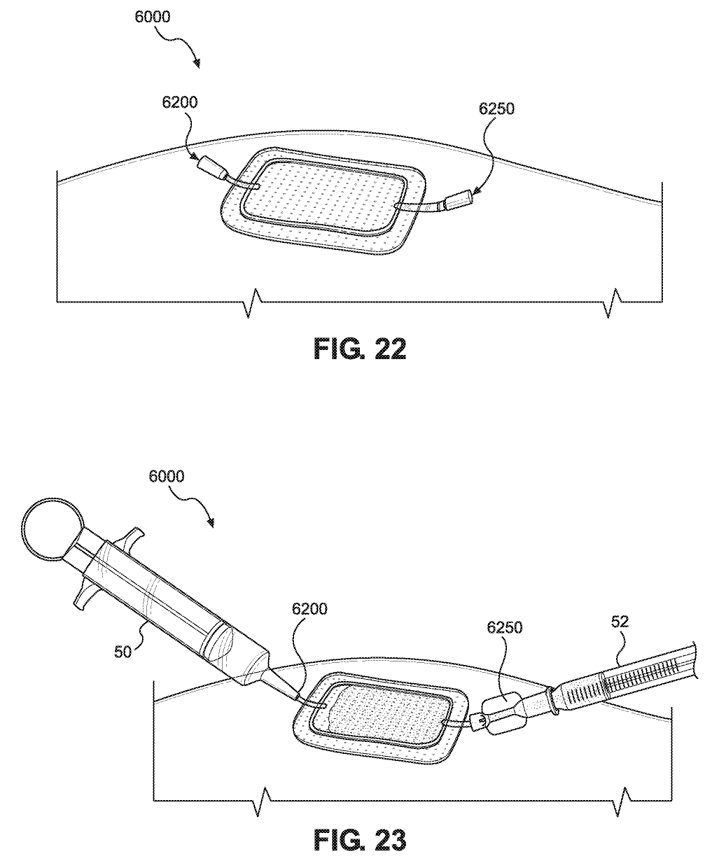

FIGS. 22 and 23 illustrate a sixth embodiment of a wound dressing 6000. The wound dressing 6000 may have one or more of the same features as the previously disclosed embodiments of the wound dressing 1000, 1000’, 3000, 4000, and/or 4000’, the entire disclosures of which is incorporated herein for sake of brevity. For example, the wound dressing 6000 may include features of the wound dressing 1000 (as discussed with reference to FIGS. 1-11) and/or the wound dressing 2000 (as discussed with reference to FIGS. 16-17). The wound dressing 6000 may be used in the method 500, as discussed.

As further illustrated, the wound dressing 6000 may have a first inlet port 6200 and a second inlet port 6250, each in communication with a chamber and/or area proximal of the wound dressing 6000. The first inlet port 6200 may be configured to be coupled to a first syringe 50, and the second inlet port 6250 may be configured to be coupled to a second syringe 52. For example, the first syringe 50 may be configured to deliver the biomaterial through the wound dressing 600. The second syringe 52 may be configured to pull fluid and/or air through the wound dressing 600, for example as the biomaterial is injected.

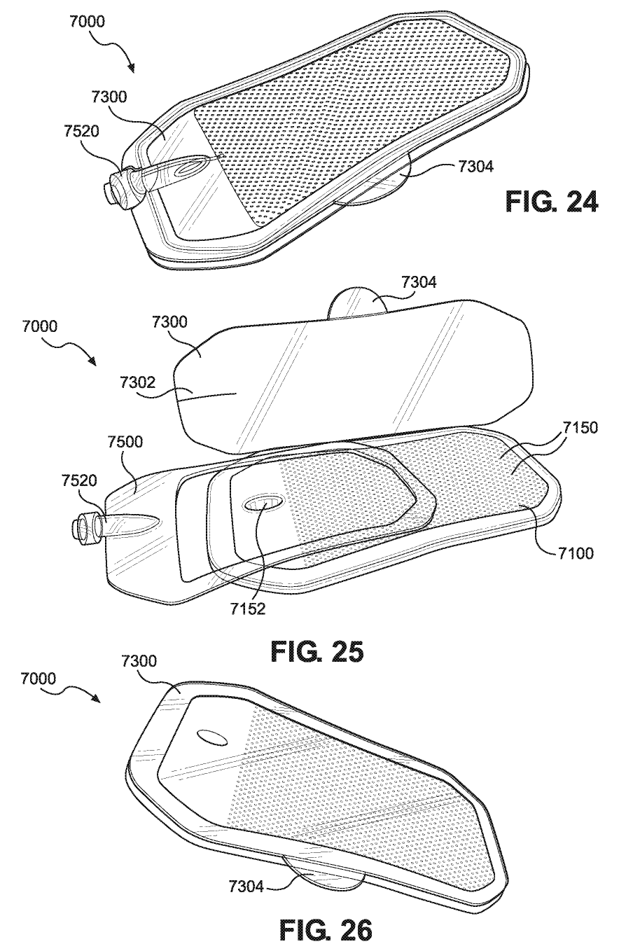

FIGS. 24-26 illustrate a seventh embodiment of a wound dressing 7000. The wound dressing 7000 may have one or more of the same features as the previously disclosed embodiments of the wound dressing 1000, 1000’, 3000, 4000, 4000’, and/or 6000, the entire disclosures of which is incorporated herein for sake of brevity. The wound dressing 7000 may be used in the method 500, as discussed.

The wound dressing 7000 may include a housing 7100, a cover 7300, and an insert 7500. The housing 7100 may include a plurality of first pores 7150 and a cavity (not shown), for example as discussed with reference to the wound dressing 300 of FIGS. 16 and 17, the entirely disclosure of which is incorporated herein for sake of brevity. The insert 7500 may be positioned between the housing 7100 and the cover 7300. The insert 7500 may be releasably coupled to the housing 7100. The insert 7500 may include an injection port 7520 configured to be in communication with an opening 7152 of the housing 7100 to provide access to the cavity of the housing 7100. The cover 7300 may include a slit 7302 configured to accommodate the injection port 7520 for access to the injection port 7520 with the cover 7300 placed over the housing 7100.

As illustrated in FIGS. 24, the injection port 7520 may be accessed through the cover 7300 for injection of the biomaterial with a container (e.g., a syringe). For example, the injection port 7520 may include a Luer lock or Luer slip configured to connect to a container (e.g., a syringe). Before, during and/or after the biomaterial is injected into the opening 7152, the cover 7300 may be lifted from the housing 7100. For example, the cover 7300 may be attached to the housing 7100 on one side to provide a pivotable connection between the cover 7300 and the housing 7100. In some embodiments, the cover 7300 may be kept open for a period of time to expose the first pores 7150 to ventilate the cavity (e.g., to promote the transition of biomaterial within cavity from a liquid state to a gel state), as discussed herein. The insert 7500 may have one or more pores and/or a complete cutout exposing the first pores of the housing 7100 to the environment. A valve (not shown) may be configured prevent backflow of the biomaterial. The valve may be attached to the housing 7100, the injection port 7520 and/or the insert 7500. In some embodiments, the valve may be attached to the housing 7100 and be configured to close the opening 7152 after removal of the insert 7500, as discussed above with respect to the wound dressing 1000 (the entire disclosure of which is incorporated herein by reference). In some embodiments, the cover 7500 may be placed back over the housing 7100, for example to close the pores 7150 and/or the opening 7152.

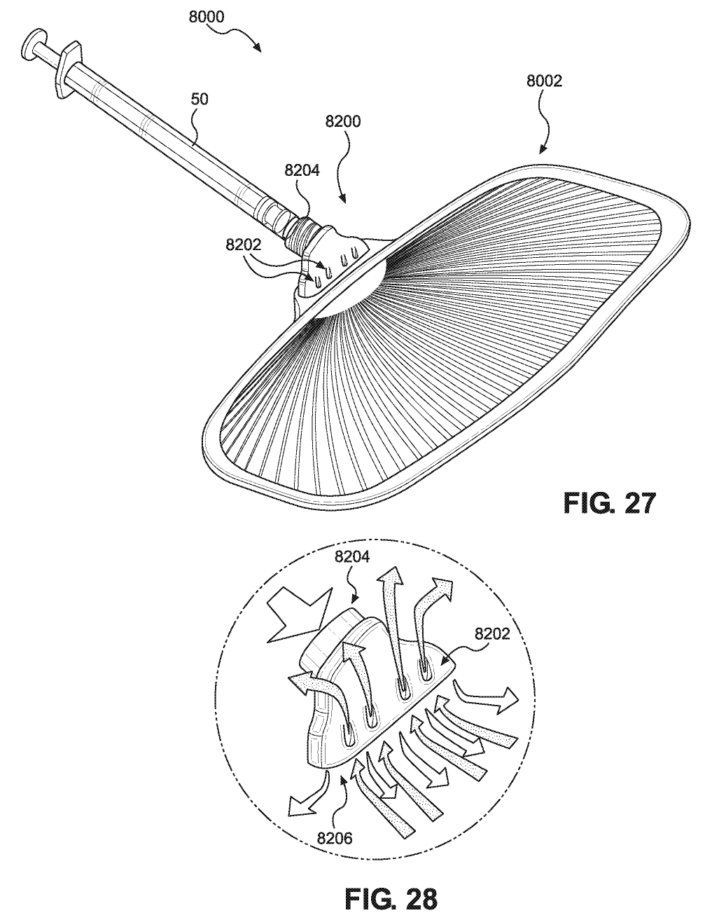

FIGS. 27 and 28 illustrate a sixth embodiment of a wound dressing 8000. The wound dressing 6000 may have one or more of the same features as the previously disclosed embodiments of the wound dressing 1000, 1000’, 3000, 4000, 4000’, 6000, and/or 7000 the entire disclosure of which is incorporated herein for sake of brevity. The wound dressing 8000 may be used in the method 500, as discussed.

As further illustrated, the wound dressing 8000 may have a bandage 8002 and an injection port 8200. The bandage 8002 and/or the injection port 8200 may have one or more features of the injection port of the other embodiments as disclosed herein, the entire disclosure of which is incorporated herein. The injection port 8200 may have an inlet 8204 configured to be coupled to a container of biomaterial to receive the biomaterial. The injection port 8200 may further have a plurality of pores 8202 sized to vent gas (e.g., air). The inlet assembly 8200 may allow ventilation of the gas through the pores 8202 during and/or after the biomaterial is introduced into and/or through the wound dressing, as discussed herein. The pores 8202 may replace or supplement pores of the bandage 8002. The ventilation features of the injection port 8200 may be implemented in any of the previously disclosed embodiments.

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

1. A wound dressing for applying a biomaterial to a subject, the wound dressing comprising:a first layer comprising a first opening and a plurality of first pores having a first width;
a second layer comprising a second opening and one or more second pores having a second width;_and an injection port attached to a distal surface of the second layer with a first adhesive and a second adhesive, wherein the first adhesive is spaced apart from the second adhesive,wherein the first layer is configured to be between the second layer and the subject when the wound dressing is applied to the subject, the first opening is at least partially aligned with the second opening to allow passage of the biomaterial from the injection port through the first layer and the second layer, and the second width is larger than the first width.

2. The wound dressing of claim 1, wherein at least a subset of the first pores is at least partially aligned with the one or more second pores to allow for ventilation through the first layer and the second layer.

3. The wound dressing of claim 1, wherein the distal surface of the second layer is at least partially uncovered to allow for ventilation through the second layer directly to the atmosphere.

4. The wound dressing of claim 3, wherein the one or more second pores are uncovered distally and directly open to the atmosphere.

5. The wound dressing of claim 1, wherein the first width is from about 0.2 microns to about 500 microns, inclusive.

6. The wound dressing of claim 1, wherein the second width is from about 1 mm to about 10 mm, inclusive.

7. The wound dressing of claim 1, further comprising a valve configured for one- way passage of the biomaterial through the first opening and/or the second opening.

8. The wound dressing of claim 1, further comprising an adhesive on a proximal surface of the second layer.

9. The wound dressing of claim 8, further comprising a non-adhesive insert positioned between the adhesive and the first layer, wherein the non-adhesive insert is configured to facilitate release of the second layer from the first layer.

10. (canceled)

11. The wound dressing of claim 1, wherein the first adhesive is at least partially around the second opening.

12. The wound dressing of claim 1, wherein the injection port has a tab adhered to the distal surface of the second layer with the second adhesive.

13. A kit comprising:
the wound dressing of claim 1; and
a container configured to form or inject the biomaterial.

14. The kit of claim 13, wherein the biomaterial comprises a platelet rich plasma (PRP) or a platelet rich fibrin matrix (PRFM).

15. A wound dressing for applying a biomaterial to a subject, the wound dressing comprising:a first layer comprising a first opening and a plurality of first pores having a first width;a second layer comprising a second opening, one or more second pores having a second width, and a flap configured to pivot relative to the second opening; andan injection port releasably attached to a distal surface of the second layer with a first adhesive and a second adhesive, wherein the first adhesive and the second adhesive are spaced apart, and the injection port is configured to introduce the biomaterial through the first opening and the second opening,
wherein the first layer is configured to be between the second layer and the subject when the wound dressing is applied to the subject, the first opening is at least partially aligned with the second opening, the flap are configured to provide one-way passage of the biomaterial through the second opening, the second width is larger than the first width, and the one or more second pores are uncovered distally and directly open to the atmosphere.

16. The wound dressing of claim 15, wherein each of the first adhesive and the second adhesive has a distal surface configured to attach to the injection port and a proximal surface configured to attach to the distal surface of the second layer, and the distal surface has a greater tackiness than the proximal surface to allow for release from the distal surface of the second layer with the injection port.

17. The wound dressing of claim 15, wherein the injection port has a tab, and the second adhesive attaches the tab to the second layer.

18. A kit comprising:
the wound dressing of claim 15; and
a container configured to form or inject the biomaterial.

19. The kit of claim 18, wherein the biomaterial comprises a platelet rich plasma (PRP) or a platelet rich fibrin matrix (PRFM).

20. A method comprising:applying a wound dressing at least partially over a wound; injecting a biomaterial, via an injection port attached to a distal surface of the wound dressing with an adhesive, through a first opening of a first layer of the wound dressing and a second opening of a second layer of the wound dressing to the wound, wherein air is vented through a plurality of first pores of the first layer and one or more second pores of the second layer;andremoving the injection port along with the adhesive from the distal surface of the wound dressing after injecting the biomaterial.

21. The method of claim 20, wherein the injecting is through a one-way valve of the wound dressing.

22-23. (canceled)

24. The method of claim 20, wherein the removing the injection port comprises lifting a tab of the injection port.

25. The method of claim 20, wherein the biomaterial comprises platelet rich plasma (PRP) or platelet rich fibrin matrix (PRFM).

26. The method of claim 20, further comprising removing the second layer from the first layer after removing the injection port from the wound dressing.

27. The method of claim 20, wherein the adhesive is a second adhesive, and the second adhesive is removed from the distal surface of the wound dressing along with the injection port.

28. A wound dressing for applying a biomaterial to a subject, the wound dressing comprising:a first layer comprising a first opening and a plurality of first pores having a first width;a second layer comprising a second opening and one or more second pores having a second width;an adhesive on a proximal surface of the second layer; andan insert positioned between the adhesive and the first layer,wherein the first layer is configured to be between the second layer and the subject when the wound dressing is applied to the subject, the first opening is at least partially aligned with the second opening to allow passage of the biomaterial through the first layer and the second layer, the second width is larger than the first width, and the insert is configured to facilitate release of the second layer from the first layer.

29. The wound dressing of claim 28, wherein the insert is positioned at a corner of the first layer.

30. The wound dressing of claim 28, wherein the insert extends laterally outside of a perimeter of the first layer.

31. The wound dressing of claim 28, wherein the second layer has an indicia indicating a peeling direction of the second layer.

32. The wound dressing of claim 28, wherein the insert is non-adhesive.
